(19) United States
(12) Patent Application Publication
(10) Pub. No.: US 2025/0375475 A1
(43) Pub. Date: Dec. 11, 2025

(54) PHARMACEUTICAL COMPOSITION AND METHODS FOR MODULATING INFLAMMATION AND OXIDATIVE STRESS

(71) Applicant: ISM BIOSCIENCES LLC, Miami, FL (US)

(72) Inventors: Max Temnik, Miami, FL (US); Oleksandr Balakin, Dnepropetrovsk (UA); Victor Gurin, Richmond, VA (US); Sergey Gurin, Richmond, VA (US)

(73) Assignee: ISM BIOSCIENCES LLC, Miami, FL (US)

(21) Appl. No.: 18/739,957

(22) Filed: Jun. 11, 2024

(51) Int. Cl. A61K 33/38; A61K 31/01; A61K 31/05; A61K 31/12; A61K 31/122; A61K 31/167; A61K 31/192; A61K 31/196; A61K 31/355; A61K 31/375; A61K 31/40; A61K 31/405; A61K 31/407; A61K 31/421; A61K 31/522; A61K 31/5415; A61K 31/593; A61K 31/616; A61K 33/04; A61K 33/06; A61K 33/20; A61K 33/22; A61K 33/24; A61K 33/26; A61K 33/30; A61K 33/34; A61K 33/44; A61K 38/06; A61K 45/06

(52) U.S. Cl. CPC A61K 33/38; A61K 31/01; A61K 31/05; A61K 31/12; A61K 31/122; A61K 31/167; A61K 31/192; A61K 31/196; A61K 31/355; A61K 31/375; A61K 31/40; A61K 31/405; A61K 31/407; A61K 31/421; A61K 31/522; A61K 31/5415; A61K 31/593; A61K 31/616; A61K 33/04; A61K 33/06; A61K 33/20; A61K 33/22; A61K 33/24; A61K 33/26; A61K 33/30; A61K 33/34; A61K 33/44; A61K 38/063; A61K 45/06

(57) ABSTRACT

Disclosed are therapeutic compositions of matter for use in regulation of local and systemic inflammation and reduction of oxidative stress to enable discovery and development of more effective pharmaceutical agents, organic compounds, and other medicinal formulations.

### FIELD

This disclosure relates to use of enriched stable isotopes in pharmaceuticals and therapeutics.

### BACKGROUND

Inflammation and oxidative stress are closely intertwined biological processes that play critical roles in the pathogenesis of a wide range of diseases. For example, several studies provide strong evidence that chronic inflammation is one of the most important pathophysiological components of synucleinopathies and taupathias, including Alzheimer's Disease (AD.) Leukograms of patients with AD reveal an increased number of monocytes and neutrophils and a low lymphocyte count. Escalated levels of monocytes and neutrophils are hallmarks of chronic inflammation and can be both precursor to AD and its consequence. A low number of lymphocytes specifies that the body's resistance to the fight infection is significantly reduced. The inflammatory responses also play decisive roles at different stages of oncological tumor development, including initiation, promotion, malignant conversion, invasion, and metastasis. Inflammation also affects immune surveillance and responses to therapy. Oxidative stress affects different signaling pathways, including growth factors and mitogenic pathways, and affects many cellular processes, including cell proliferation, and thus stimulates the uncontrolled growth of cancerous cells, which encourages the development of tumors and begins the process of carcinogenesis.

Inflammation is the body's natural response to harmful stimuli, such as pathogens, damaged cells, or irritants. It is a protective mechanism involving immune cells, blood vessels, and molecular mediators that aim to eliminate the initial cause of cell injury, clear out necrotic cells and tissues damaged from the original insult and the inflammatory process, and initiate tissue repair. While reactive acute inflammation is natural, beneficial, and necessary process for maintaining a healthy organism, chronic inflammation can render several detrimental effects. This harmful impact arises because the immune system, in a state of chronic inflammation, can mistakenly target healthy tissues and organs, leading to a variety of health issues and diseases.

Oxidative stress refers to a state where there is an imbalance between the production of reactive oxygen species (“ROS”) and the body's ability to counteract their harmful effects through neutralization by antioxidants. ROS are chemically reactive molecules containing oxygen, which include free radicals and peroxides. While ROS are essential for several biological processes, excessive ROS can lead to cellular damage, affecting lipids, proteins, and DNA. The relationship between oxidative stress and inflammation is dynamic and bidirectional. Oxidative stress can trigger inflammatory pathways, while inflammation can lead to increased oxidative stress, creating a vicious cycle that contributes to the pathology of various diseases.

Chronic inflammation and oxidative stress have been linked to a wide range of diseases that collectively represent the leading causes of disability and mortality worldwide. These include cardiovascular, metabolic, neurodegenerative, autoimmune, oncological, and other diseases and disorders. One of the key factors contributing to chronic inflammation is the body's response to certain lifestyle, environmental, and genetic factors. These can include poor diet, physical inactivity, smoking, and exposure to environmental pollutants. Understanding the mechanisms linking oxidative stress and inflammation has significant implications for the development of therapeutic strategies. Antioxidants, which can neutralize ROS, and anti-inflammatory drugs are commonly used to treat conditions associated with oxidative stress and inflammation.

However, the challenge remains in effectively targeting these therapies to break the cycle of oxidative stress and inflammation without disrupting their normal physiological functions.

### SUMMARY

This disclosure relates to the role of isotopic ratios of essential chemical elements in physiological processes in mammals.

In one aspect, this disclosure provides a pharmaceutical composition for modulating inflammation and oxidative stress in mammals including at least one chemical element selected from H, Li, C, B, N, O, Mg, Si, S, K, Cl, Ca, V, Cr, Fe, Ni, Cu, Zn, Sr, Mo, Se, Ge, Ga, Br, Rb, Ag, and Ba, or a combination thereof, wherein said chemical elements are altered from their natural sample state so that ¹H is enriched to exceed 99.985%, ⁶Li is enriched to exceed 10.06%, ¹²C is enriched to exceed 98.93%, ¹⁰B is enriched to exceed 21.01%, ¹⁴N is enriched to exceed 99.64%, ¹⁶O is enriched to exceed 99.81%, ¹⁷O is enriched to exceed 0.01%, ²⁴Mg is enriched to exceed 80.01%, ²⁵Mg is enriched to exceed 10.01%, ²⁸Si is enriched to exceed 92.51%, ²⁹Si is enriched to exceed 5.01%, ³²S is enriched to exceed 95.03%, ³³S is enriched to exceed 0.91%, ³⁹K is enriched to exceed 93.51%, ³⁵Cl is enriched to exceed 76.01%, ⁴⁰Ca is enriched to exceed 96.95%, ⁴²Ca is enriched to exceed 0.65%, ⁴³Ca is enriched to exceed 0.65%, ⁵⁰V is enriched to exceed 0.25%, ⁵⁰Cr is enriched to exceed 4.25%, ⁵²Cr is enriched to exceed 85.01%, ⁵⁴Fe is enriched to exceed 5.85%, ⁵⁶Fe is enriched to exceed 91.8%, ⁵⁸Ni is enriched to exceed 68.10%, ⁶⁰Ni is enriched to exceed 26.51%, ⁶¹Ni is enriched to exceed 1.12%, ⁶³Cu is enriched to exceed 69.10%, ⁶⁴Zn is enriched to exceed 58.61%, ⁶⁶Zn is enriched to exceed 28.10%, ⁸⁴Sr is enriched to exceed 0.60%, ⁹²Mo is enriched to exceed 15.01%, ⁹⁴Mo is enriched to exceed 9.25%, ⁹⁵Mo is enriched to exceed 15.95%, ⁹⁶Mo is enriched to exceed 16.69%, ⁷⁴Se is enriched to exceed 0.90%, ⁷⁶Se is enriched to exceed 9.25%, ⁷⁷Se is enriched to exceed 7.65%, ⁷⁸Se is enriched to exceed 23.81%, ⁷⁰Ge is enriched to exceed 20.55%, ⁷²Ge is enriched to exceed 27.57%, ⁷³Ge is enriched to exceed 7.77%, ⁶⁹Ga is enriched to exceed 64.11%, ⁷⁹Br is enriched to exceed 50.70%, ⁸⁵Rb is enriched to exceed 72.20%, ¹⁰⁷Ag is enriched to exceed 51.61%, ¹³⁰Ba is enriched to exceed 0.12%, ¹³²Ba is enriched to exceed 0.12%, ¹³⁴Ba is enriched to exceed 2.42%, ¹³⁵Ba is enriched to exceed 6.60%, and/or ¹³⁶Ba is enriched to exceed 7.86%. Therapeutic administration of these stable isotopes modulates the levels of inflammation and oxidative stress, hence rendering a therapeutic effect on several vital biological processes. These biological processes can be improved by reducing the inflammation and oxidative stress, which is achievable by administering these stable isotopes on an isotope-selective basis.

In another aspect, this disclosure provides a method of modulating local and/or systemic inflammation in mammals including administering a therapeutically effective amount to a subject in need thereof of at least one isotope-specific chemical element selected from ⁶Li, ¹⁰B, ²⁴Mg, ²⁵Mg, ²⁸Si, ²⁹Si, ³²S, ³³S, ³⁹K, ³⁵Cl, ⁴⁰Ca, ¹²Ca, ⁴³Ca, ⁵⁰V, ⁵⁰Cr, ⁵²Cr, ⁵⁴Fe, ⁵⁶Fe, ⁵⁸Ni, ⁶⁰Ni, ⁶¹Ni, ⁶³Cu, ⁶⁴Zn, ⁶⁶Zn, ⁸⁴Sr, ⁹²Mo, ⁹⁴Mo, ⁹⁵Mo, ⁹⁶Mo, ⁷⁴Se, ⁷⁶Se, ⁷⁷Se, ⁷⁸Se, ⁷⁰Ge, ⁷²Ge, ⁷³Ge, ⁶⁹Ga, ⁷⁹Br, ⁸⁵Rb, 107Ag, ¹³⁰Ba, ¹³²Ba, ¹³⁴Ba, ¹³⁵Ba, and/or ¹³⁶Ba present in elemental form, atomic form, colloidal substance form, in form of pharmaceutically acceptable compound, or in form of salt, or in combination of these forms, wherein ⁶Li is enriched to exceed 10.06%, ¹²C is enriched to exceed 98.93%, ¹⁰B is enriched to exceed 21.01%, ²⁴Mg is enriched to exceed 80.01%, ²⁵Mg is enriched to exceed 10.01%, ²⁸Si is enriched to exceed 92.51%, ²⁹Si is enriched to exceed 5.01%, ³²S is enriched to exceed 95.03%, ³³S is enriched to exceed 0.91%, ³⁹K is enriched to exceed 93.51%, ³⁵Cl is enriched to exceed 76.01%, ⁴⁰Ca is enriched to exceed 96.95%, ⁴²Ca is enriched to exceed 0.65%, ⁴³Ca is enriched to exceed 0.65%, ⁵⁰V is enriched to exceed 0.25%, ⁵⁰Cr is enriched to exceed 4.25%, ⁵²Cr is enriched to exceed 85.01%, ⁵⁴Fe is enriched to exceed 5.85%, ⁵⁶Fe is enriched to exceed 91.8%, ⁵⁸Ni is enriched to exceed 68.10%, ⁶⁰Ni is enriched to exceed 26.51%, ⁶¹Ni is enriched to exceed 1.12%, ⁶³Cu is enriched to exceed 69.10%, ⁶⁴Zn is enriched to exceed 58.61%, ⁶⁶Zn is enriched to exceed 28.10%, ⁸⁴Sr is enriched to exceed 0.60%, ⁹²Mo is enriched to exceed 15.01%, ⁹⁴Mo is enriched to exceed 9.25%, ⁹⁵Mo is enriched to exceed 15.95%, ⁹⁶Mo is enriched to exceed 16.69%, ⁷⁴Se is enriched to exceed 0.90%, ⁷⁶Se is enriched to exceed 9.25%, ⁷⁷Se is enriched to exceed 7.65%, ⁷⁸Se is enriched to exceed 23.81%, ⁷⁰Ge is enriched to exceed 20.55%, ⁷²Ge is enriched to exceed 27.57%, ⁷³Ge is enriched to exceed 7.77%, ⁶⁹Ga is enriched to exceed 64.11%, ⁷⁹Br is enriched to exceed 50.70%, ⁸⁵Rb is enriched to exceed 72.20%, ¹⁰⁷Ag is enriched to exceed 51.61%, ¹³⁰Ba is enriched to exceed 0.12%, ¹³²Ba is enriched to exceed 0.12%, ¹³⁴Ba is enriched to exceed 2.42%, ¹³⁵Ba is enriched to exceed 6.60%, and/or ¹³⁶Ba is enriched to exceed 7.86%. In another aspect, this disclosure provides a method of reducing oxidative stress including administering a therapeutically effective amount to a subject in need thereof of at least one isotope-specific chemical element selected from of 6Li, 12C, 10B, 24Mg, 25Mg, 28Si, 29Si, 32S, 33S, 39K, 35Cl, 40Ca, 42Ca, 43Ca, 50V, 50Cr, 52Cr, 54Fe, 56Fe, 58Ni, 60Ni, 61Ni, 63Cu, 64Zn, 66Zn, 84Sr, 92Mo, 94Mo, 95Mo, 96Mo, 74Se, 76Se, 77Se, 78Se, 70Ge, 72Ge, 73Ge, 69Ga, 79Br, 85Rb, 107Ag, 130Ba, 132Ba, 134Ba, 135Ba, and/or 136Ba, present in elemental form, atomic form, colloidal substance form, in form of pharmaceutically acceptable compound, or in form of a salt, wherein 6Li is enriched to exceed 10.06%, 12C is enriched to exceed 98.93%, 10B is enriched to exceed 21.01%, 24Mg is enriched to exceed 80.01%, 25Mg is enriched to exceed 10.01%, 28Si is enriched to exceed 92.51%, 29Si is enriched to exceed 5.01%, 32S is enriched to exceed 95.03%, 33S is enriched to exceed 0.91%, 39K is enriched to exceed 93.51%, 35Cl is enriched to exceed 76.01%, 40Ca is enriched to exceed 96.95%, 42Ca is enriched to exceed 0.65%, 43Ca is enriched to exceed 0.65%, 50V is enriched to exceed 0.25%, 50Cr is enriched to exceed 4.25%, 52Cr is enriched to exceed 85.01%, 54Fe is enriched to exceed 5.85%, 56Fe is enriched to exceed 91.8%, 58Ni is enriched to exceed 68.10%, 60Ni is enriched to exceed 26.51%, 61Ni is enriched to exceed 1.12%, 63Cu is enriched to exceed 69.10%, 64Zn is enriched to exceed 58.61%, 66Zn is enriched to exceed 28.10%, 84Sr is enriched to exceed 0.60%, 92Mo is enriched to exceed 15.01%, 94Mo is enriched to exceed 9.25%, 95Mo is enriched to exceed 15.95%, 96Mo is enriched to exceed 16.69%, 74Se is enriched to exceed 0.90%, 76Se is enriched to exceed 9.25%, 77Se is enriched to exceed 7.65%, 78Se is enriched to exceed 23.81%, 70Ge is enriched to exceed 20.55%, 72Ge is enriched to exceed 27.57%, 73Ge is enriched to exceed 7.77%, 69Ga is enriched to exceed 64.11%, 79Br is enriched to exceed 50.70%, 85Rb is enriched to exceed 72.20%, 107Ag is enriched to exceed 51.61%, 130Ba is enriched to exceed 0.12%, 132Ba is enriched to exceed 0.12%, 134Ba is enriched to exceed 2.42%, 135Ba is enriched to exceed 6.60%, and/or 136Ba is enriched to exceed 7.86%.

### BRIEF DESCRIPTION OF DRAWINGS

FIG. 1A and FIG. 1B illustrate reduced inflammation as evidenced by key neuroinflammatory biomarker GFAP expressed by astrocytes after administering light stable isotope ⁶⁴Zn in combination with administration of L-Aspartate amino acid. FIG. 1A and FIG. 1B show a significant reduction in GFAP in both blood (FIG. 1A) and the brain and periphery (FIG. 1B), signaling a return to homeostasis. GFAP is an intermediate filament protein predominantly expressed by astrocytes. It serves as a marker of astrocyte activation, also known as astrogliosis, which is a common response to central nervous system (CNS) injury and inflammation. Elevated levels of GFAP in blood or cerebrospinal fluid (CSF) indicate reactive astrogliosis, which is often associated with neuroinflammatory processes. Administration of stable light isotope ⁶⁴Zn with L-aspartic acid has lowered the inflammation significantly as indicated by lowered GFAP levels.

FIG. 2 illustrates reduced inflammation as evidenced by astroglia, which play a crucial role in regulating inflammation within the central nervous system (CNS). Astrocytes can release a variety of pro-inflammatory cytokines (e.g., IL-1β, IL-6, TNF-α), chemokines, and reactive oxygen species (ROS), which contribute to the inflammatory response. Administration of stable light isotope ⁶⁴Zn with L-aspartic acid has lowered the inflammation significantly as evidenced by astroglia.

FIG. 3 shows normalization of the absolute number of circulating leukocytes after administering light stable isotope ⁶⁴Zn and L-aspartate amino acid to rodents. The number of circulating leukocytes can provide valuable information about the presence and severity of inflammation in the body. An increase in the number of circulating leukocytes, known as leukocytosis, is a common indicator of an ongoing inflammatory process. During inflammation, the bone marrow is stimulated to release more WBCs into the bloodstream to combat the inflammatory stimulus, such as infection, injury, or autoimmune disorders. The number and types of circulating leukocytes play a crucial role in the inflammatory response by participating in the immune defense, migrating to sites of inflammation, and serving as biomarkers for the presence and severity of inflammatory conditions. Administration of stable light isotope ⁶⁴Zn with L-aspartic acid has lowered the inflammation significantly as indicated by reduced number of circulating leukocytes.

FIG. 4 shows changes in relative number of CD86+circulating phagocytes in Aβ1-40 rat models of Alzheimer's disease (AD), which indicates reduction of inflammation after administering light stable isotope ⁶⁴Zn and L-aspartic amino acid. CD86 is a co-stimulatory molecule that is involved in the activation of T cells and the modulation of immune responses. The number of CD86+microglia is a critical factor in the inflammatory processes of Alzheimer's disease. Increased CD86 expression in aged and Aβ-treated microglia suggests a heightened inflammatory state and a potential for enhanced immune interactions, which can contribute to the pathogenesis and progression of AD. Administration of stable light isotope ⁶⁴Zn with L-aspartic acid has lowered the inflammation significantly as indicated by reduced CD86 expression.

FIG. 5 shows reduction CD206 expression, which indicates reduction of inflammation after administering light stable isotope ⁶⁴Zn and L-aspartic acid. CD206, also known as the mannose receptor, plays a significant role in the context of oxidative stress, particularly through its involvement in macrophage function and polarization. Oxidative stress, characterized by the excessive production of reactive oxygen species (ROS), can influence the polarization and function of macrophages. CD206+M2 macrophages are particularly relevant in this context as they help mitigate the damaging effects of oxidative stress by promoting anti-inflammatory responses and tissue repair mechanisms. Administration of stable light isotope ⁶⁴Zn with included L-aspartic acid has significantly reduced oxidative stress as indicated by the decrease in CD206 levels.

FIG. 6 shows reduction of reactive oxygen species in microglia after administering ⁶⁴Zn L-aspartate. Activation of NOX2 leads to the production of superoxide anions, which can further react to form other ROS such as hydrogen peroxide and hydroxyl radicals. Mitochondria in microglia also produce ROS as by-products of ATP production. Dysfunctional mitochondria can lead to excessive ROS generation. Persistent activation of microglia and excessive ROS production can lead to chronic inflammation. This is a hallmark of several diseases. Administration of stable light isotope ⁶⁴Zn with L-aspartic acid has lowered the inflammation significantly as evidenced by reduced reactive oxygen species in microglia.

FIG. 7 illustrates an effect of administering light isotope ⁶⁴Zn included with L-Aspartic amino acid on metabolic characteristics of microglia/macrophages in LPS-lesioned rats, which indicates a reduction of oxidative stress. The metabolic characteristics of microglia and macrophages are closely related to oxidative stress, particularly through their roles in energy metabolism and the production of reactive oxygen species (ROS). Administration of stable light isotope ⁶⁴Zn with L-aspartic acid has significantly reduced oxidative stress as indicated by the metabolic characteristics of microglia/macrophages.

### Description

Isotope enrichment refers to the process of increasing the proportion of a specific isotope in a mixture of isotopes. Historically, the enriched stable isotopes of carbon (C), nitrogen (N), and oxygen (O) are used in both medicine and pharmaceutical applications. These enriched isotopes are used for various purposes, including drug development and diagnostics. For instance, isotopes ¹³C and ¹⁵N have been used in clinical medicine and biological studies. They are particularly valuable in the development of diagnostic tests, such as the ¹³C urea breath test for detecting Helicobacter pylori infections. Also, the chemical compounds labeled with highly enriched ¹³C are used in breath tests for diagnosing liver and intestine diseases. Modern medicine has started to recognize the importance of metals in physiological processes. Metallomics is a recently developing interdisciplinary science that integrates chemistry, biology, physics, and environmental sciences to study the role, distribution, dynamics, and impact of metals and metalloids in biological systems. This field aims to elucidate the “what, where, when, how, and why” of inorganic elements in cells, tissues, organisms, and their environments, employing a wide range of analytical, bioinorganic, medicinal, and environmental approaches. The term “metallome” refers to the entirety of metal and metalloid species present in a biological system. Metallomics, therefore, is the comprehensive analysis of the metallome, encompassing the study of metalloproteins, metallometabolites, and other metal-containing biomolecules within cells or tissues. It addresses the complex interactions between living systems and inorganic elements, aiming to provide the systems biology solutions by describing the interlinks and connections among various pathways and processes involving metal ions in the cell.

The metals and metalloid species present in mammals are chemical elements, many of each consist of the atoms featuring the same number of electrons and protons, but different number of neutrons in their nuclei. The atoms differing by the number of neutrons are isotopes, which are distinct nuclear species of the same chemical element. Due to different numbers of neutrons, the isotopes of the same chemical element have the same atomic number and position in the periodic table but differ in nucleon numbers. The isotopes are referred to as stable and radioactive.

Radioactive isotopes have been used in pharmaceuticals since the early 1930s. One of the earliest recorded uses was by John Lawrence, who in 1936 used phosphorus-32, a radioactive isotope, to treat leukemia. This marked the first clinical therapeutic application of an artificially enriched radionuclide. The development and use of enriched radioactive isotopes were further advanced by the work of the U.S. Atomic Energy Commission after World War II, which mass-produced radioisotopes for medical use, distributing them to scientists and physicians.

The use of enriched stable isotopes in pharmaceuticals commenced with the discovery of deuterium by Harold Urey in 1932. For the first several decades, the focus was on understanding biological and chemical processes, leveraging the ability of stable isotopes like deuterium (Rita Maria Concetta Di Martino, 2023) and tritium to be detected through mass spectrometry and radioactivity measurements, respectively. Despite tritium being a radioactive isotope, its long half-life and the stable nature of deuterium allowed for safe handling and application in various biomedical areas. The concept of deuteration, substituting a hydrogen atom with deuterium, emerged as a significant advancement in drug discovery. This subtle structural modification can improve the pharmacokinetic and toxicity profiles of drugs, potentially leading to enhanced efficacy and safety. The approval of deutetrabenazine in 2017 (U.S. Pat. No. 8,524,733) as the first deuterated drug by the FDA marked a milestone, followed by the approval of deucravacitinib in 2022 (WO 2018/183656), showcasing the shift towards novel drug discovery through deuteration.

The historic use of stable isotopes in their natural (not enriched) isotopic ratios in pharmaceuticals has been primarily focused on their role in drug metabolism studies, clinical pharmacology, and personalized medicine. They are instrumental in determining the pharmacokinetic profile, bioavailability, and release profile of drug substances and delivery systems. Moreover, stable isotopes facilitate personalized medicine by enabling patient assessment in relation to specific drug treatments, thus optimizing therapeutic outcomes. (Reinout C A Schellekens, 2011). However, the prior art used stable isotopes in their natural abundances and not in an enriched form.

The term “natural abundance” refers to the distribution of isotopes of a chemical element as they are found in nature. The natural abundance of an isotope is expressed as a percentage of the total amount of the element in a sample or environment. For example, hydrogen has two stable isotopes, 1H (protium) and 2H (deuterium), with natural abundances of approximately 99.985% and 0.015%, respectively. This means that in a sample of naturally occurring hydrogen, nearly all the atoms will be protium, with a very small fraction being deuterium (Knowledge). The natural abundance of stable isotopes varies for different elements. These natural abundances can be altered in biological systems through processes known as isotopic fractionation, where lighter isotopes react or diffuse slightly faster than their heavier counterparts due to differences in mass and covalent strength.

Isotopic fractionation occurs naturally in biological organisms as they perform various metabolic functions. For example, during photosynthesis, plants preferentially incorporate the lighter 12C isotope over 13C, leading to a depletion of 13C in plant tissues compared to the atmospheric CO (Knowledge, Stable isotope ratio). Similarly, nitrogen isotopes fractionate during processes like nitrogen fixation, assimilation, and trophic transfer, providing insights into nutrient cycles and food web dynamics (Knowledge, Stable isotope ratio).

The depletion of stable isotopes from the human body is a relatively recently discovered biophysiological process that can occur under various physiological conditions and can be influenced by diet, pharmaceutical intake, metabolic processes, and environmental factors, among others. The natural abundance and fractionation of stable isotopes can be altered by disease states or physiological stress, affecting their distribution and concentration in tissues.

Studies in metallomics have shown that stable isotopes of chemical elements in a biological organism vary from their natural abundance ratios. Several recent studies have shown that certain diseases can cause pathology-influenced isotopic fractionation. This is particularly evident in conditions that affect bone turnover or collagen synthesis, where the isotopic composition of these tissues can provide insights into the physiological state of the individuals (Reitsema, 2013).

The concept of isotope depletion, particularly concerning light isotopes, in the human body revolves around the natural processes and interventions that lead to a decrease in the relative abundance of these isotopes within biological systems. This phenomenon has been observed and studied in various contexts, including nutritional studies, medical applications, and physiological research. In the human body, isotopic fractionation can occur through metabolic activities, where lighter isotopes can be preferentially utilized or excreted, leading to a relative enrichment of heavier isotopes in the cells and tissues. The exact reasons causing isotopic fractionation are yet unknown, although numerous hypotheses and theories exist (for example, the kinetics of isotopic effect.) The depletion of light isotopes can have significant implications for understanding pathological processes and developing therapeutic strategies. In this regard, the third-party research has been mainly focused on the depletion of light isotopes of carbon (C), hydrogen (H), nitrogen (N), and oxygen (O), which are absolutely abundant of light isotopes.

For example, studies have shown that the human body can fractionate hydrogen stable isotopes, with an increase in the content of the heavy hydrogen isotope (deuterium, ²H) in body fluids compared to potable water (Y Siniak, 2006). This supports the theory that the human organism eliminates heavy stable isotopes of biogenous chemical elements, suggesting a natural preference or selective process for lighter isotopes in physiological functions. The present invention introduces novel composition of matter and methods of using light stable isotopes for regulating inflammation and reducing oxidative stress by administering light stable isotopes for cellular uptake.

One of the most direct ways to influence the isotopic composition of the human body is through the consumption of water depleted of heavy isotopes, such as deuterium-depleted water (DDW) and water with reduced levels of heavy isotope ¹⁸O. Water depleted of heavy isotopes has shown numerous biological and health effects in vitro, in vivo, and in clinical settings. For instance, consumption of deuterium-depleted water has been associated with improved mitochondrial function, which is crucial for energy production in the form of adenosine triphosphate (ATP) (Z Kharaeva, 2021). This suggests that the depletion of light isotopes from the human body, through the consumption of isotopically altered water, can have significant physiological and metabolic implications. Furthermore, the isotopic composition of biological standards, such as iron isotopes, offers novel opportunities to study metabolic pathways and diseases related to metal homeostasis (Kubik, 2021).

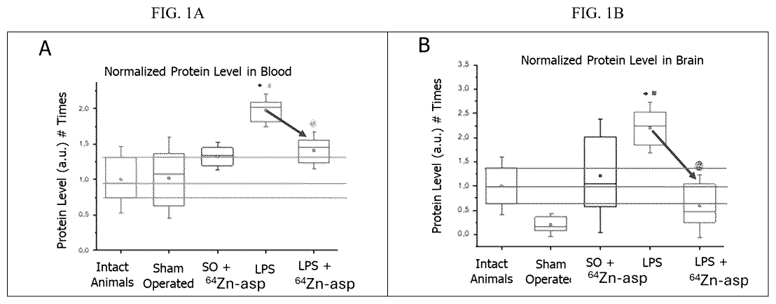

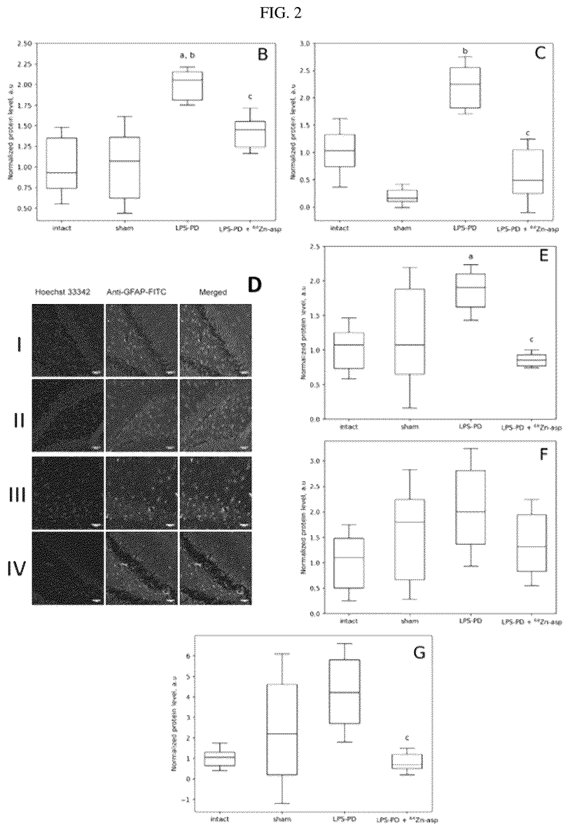

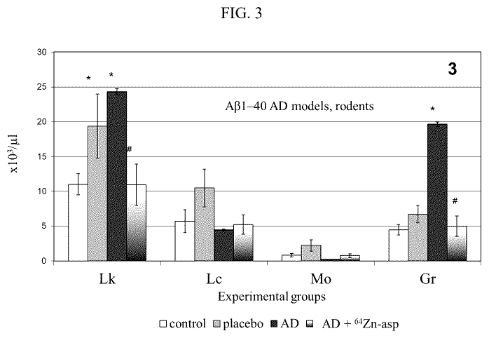

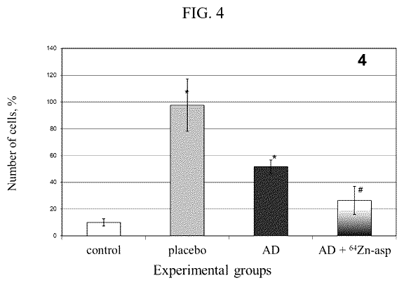

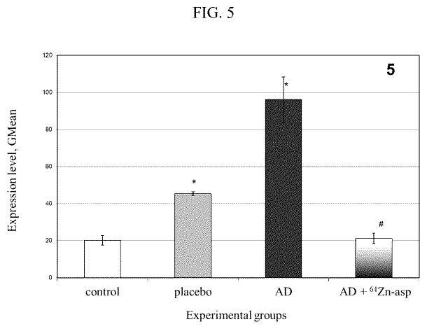

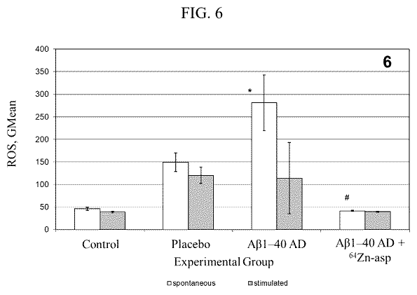

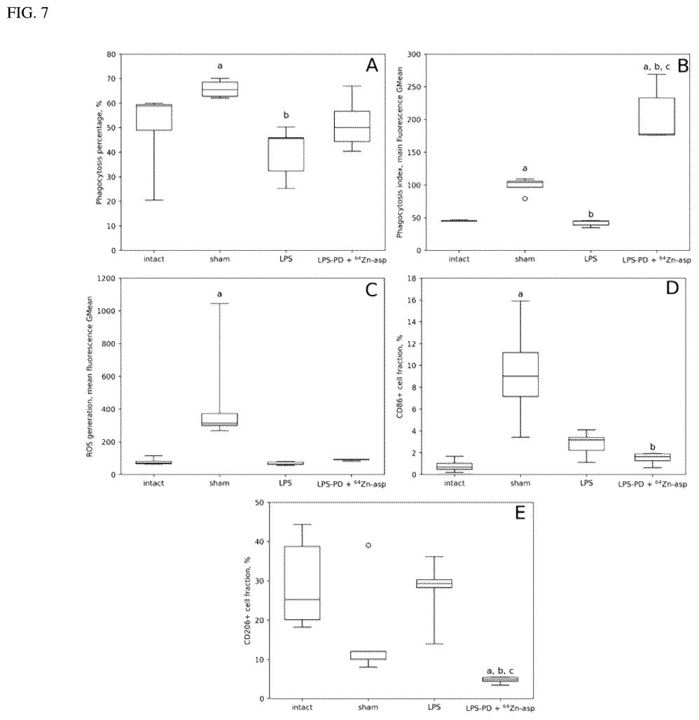

In addition to H, C, N, and O isotopes, this disclosure provides enriched isotopes of Li, B, Mg, Si, S, K, Cl, V, Cr, Fe, Ni, Cu, Zn, Sr, Mo, Se, Ge, Br, Rb, and Ba, and their inter-relationships in the development of various pathologies. The light stable isotope ⁶⁴Zn is an exemplary representative of the isotopes in the disclosed composition. The research subject substance was combined with L-aspartate, as a representative of the proteinogenic amino acids group. The effects of ⁶⁴Zn L-Aspartate on inflammation and oxidative stress both in vitro and in vivo using mouse models of various neurological, metabolic, autoimmune, cardiovascular, and oncological disorders are studied (see FIGS. 1-7). The disclosed compositions and methods reduce inflammation and oxidative stress across all these areas.

Inflammation and oxidative stress are two interrelated processes that play significant roles in the pathogenesis of various chronic diseases. Both conditions can cause extensive damage to cells, tissues, and organs, leading to a range of health issues.

The essential chemical elements, also known as essential elements or essential nutrients, are chemical elements required by living organisms for their proper structure and function. These elements must be obtained from the diet or environment because the mammal organism cannot synthesize them in sufficient quantities. Each essential element features one or more stable isotopes (atoms.) Different from radioactive atoms, stable atoms are specific forms of an element (nuclides) that do not undergo radioactive decay. The essential elements deplete from mammal organisms as a result of exposure to dietary and environmental factors, hence causing various deficiencies that are harmful to healthy biological functions.

The relationship between metallome deficiencies and inflammation and oxidative stress is complex and multifaceted. Metallome is generally referred to as the set of metal ions within a biological system. Correlation analyses have shown robust associations between cytokines and metal ions, indicating that metal homeostasis can influence inflammatory responses during several disease pathogenesis. Redox-active metals like iron (Fe) and copper (Cu) can undergo redox cycling reactions, producing reactive oxygen species (ROS) such as superoxide anion and hydroxyl radicals. These ROS can cause significant damage to DNA, proteins, and lipids, leading to various diseases including cancer, cardiovascular diseases, and neurological disorders. At the same time, metallome deficiencies and imbalances can significantly influence inflammation and oxidative stress through various mechanisms, including redox cycling, ROS production, and disruption of metal homeostasis. In summary, the metallome significantly influences inflammation and oxidative stress through various mechanisms, including redox cycling, ROS production, and disruption of metal homeostasis. Understanding these relationships is crucial for developing therapeutic strategies to mitigate metal-induced health effects. The experiments conducted by the named inventors have been focused on understanding these relationships and investigating the novel mechanisms for reducing inflammation and oxidative stress. Specifically, the inventors have focused on the role of isotopic fractionation in modulating the levels of inflammation and oxidative stress and use of the isotopes cited in the claims.

Stable isotope ⁶⁴Zinc is selected as representative of metallome element zinc, which plays a unique role as a redox-inert metal that can protect against inflammation and oxidative stress by stabilizing proteins and membranes and supporting the immune system. The ⁶⁴Zn isotope is selected also because of its important role in protein synthesis through ribosomal function. L-aspartate, also known as L-aspartic acid, is selected as a representative of organic acids that play a crucial role in various metabolic processes in the human body, including its central role in cell proliferation.

Stable isotopes are atoms of the same chemical element which defer primarily in their mass due to having different numbers of neutrons in their nuclei. Zinc atoms have five stable isotopes of which ⁶⁴Zn is the lightest, followed by ⁶⁶Zn. L-aspartate is one of the 22 proteinogenic amino acids, meaning it is directly incorporated into proteins during translation. It is encoded by the codons GAU and GAC and is involved in several key metabolic pathways. It also acts as a neurotransmitter, stimulating NMDA receptors.

The disclosed pharmaceutical composition includes specific enriched light isotopes in their elemental form, and also in colloidal substance form, or in form of a pharmaceutically acceptable salt made with any organic or inorganic acid, inclusive of and not limited to an amino acid, or a subclass or a variation of an amino acid, an ethylenediaminetetraacetic acid, a citric acid, a diethylenetriaminepentaacetic acid, an ethyleneglycol-bis(2-aminoethyl)-N,N,N′,N′-tetraacetic acid, or a combination thereof. For example, without limitation, a proteinogenic amino acid (such as L-Aspartate) can be combined with ²⁸Mg and ⁶³Cu isotopes. However, the isotopes do not necessarily have to be administered as part of a pharmaceutically acceptable salt of an organic or inorganic acid. Instead, these acids can be administered in combination with the isotopes (for example as parts of an injection or infusion solution) without forming the salt prior to the combination.

In one aspect, this disclosure provides a pharmaceutical composition for modulating inflammation and oxidative stress in mammals including at least one chemical element selected from H, Li, C, B, N, O, Mg, Si, S, K, Cl, Ca, V, Cr, Fe, Ni, Cu, Zn, Sr, Mo, Se, Ge, Ga, Br, Rb, Ag, and Ba, or a combination thereof, wherein said chemical elements are altered from their natural sample state so that ¹H is enriched to exceed 99.985%, ⁶Li is enriched to exceed 10.06%, ¹²C is enriched to exceed 98.93%, ¹⁰B is enriched to exceed 21.01%, ¹⁴N is enriched to exceed 99.64%, ¹⁶O is enriched to exceed 99.81%, ¹⁷O is enriched to exceed 0.01%, ²⁴Mg is enriched to exceed 80.01%, ²⁵Mg is enriched to exceed 10.01%, ²⁸Si is enriched to exceed 92.51%, ²⁹Si is enriched to exceed 5.01%, ³²S is enriched to exceed 95.03%, ³³S is enriched to exceed 0.91%, ³⁹K is enriched to exceed 93.51%, ³⁵Cl is enriched to exceed 76.01%, ⁴⁰Ca is enriched to exceed 96.95%, ⁴²Ca is enriched to exceed 0.65%, ⁴³Ca is enriched to exceed 0.65%, ⁵⁰V is enriched to exceed 0.25%, ⁵⁰Cr is enriched to exceed 4.25%, ⁵²Cr is enriched to exceed 85.01%, ⁵⁴Fe is enriched to exceed 5.85%, ⁵⁶Fe is enriched to exceed 91.8%, ⁵⁸Ni is enriched to exceed 68.10%, ⁶⁰Ni is enriched to exceed 26.51%, ⁶¹Ni is enriched to exceed 1.12%, ⁶³Cu is enriched to exceed 69.10%, ⁶⁴Zn is enriched to exceed 58.61%, ⁶⁶Zn is enriched to exceed 28.10%, ⁸⁴Sr is enriched to exceed 0.60%, ⁹²Mo is enriched to exceed 15.01%, ⁹⁴Mo is enriched to exceed 9.25%, ⁹⁵Mo is enriched to exceed 15.95%, ⁹⁶Mo is enriched to exceed 16.69%, ⁷⁴Se is enriched to exceed 0.90%, ⁷⁶Se is enriched to exceed 9.25%, ⁷⁷Se is enriched to exceed 7.65%, ⁷⁸Se is enriched to exceed 23.81%, ⁷⁰Ge is enriched to exceed 20.55%, ⁷²Ge is enriched to exceed 27.57%, ⁷³Ge is enriched to exceed 7.77%, ⁶⁹Ga is enriched to exceed 64.11%, ⁷⁹Br is enriched to exceed 50.70%, ⁸⁵Rb is enriched to exceed 72.20%, ¹⁰⁷Ag is enriched to exceed 51.61%, ¹³⁰Ba is enriched to exceed 0.12%, ¹³²Ba is enriched to exceed 0.12%, ¹³⁴Ba is enriched to exceed 2.42%, ¹³⁵Ba is enriched to exceed 6.60%, and/or ¹³⁶Ba is enriched to exceed 7.86%. The isotopes can be included in the disclosed pharmaceutical composition in elemental or atomic form, both of which (not mutually exclusive and not mutually inclusive) can be used individually or as a combination of two or more cited isotopes, or as a component of a pharmaceutical compound.

In some embodiments, a disclosed pharmaceutical composition includes (in addition to the enriched isotopes) at least one of a carboxylic acid, a sulfonic acid, a dicarboxylic acid, a hydroxy acid, an amino acid, a fatty acid, an aromatic acid, a keto acid, a thiol, an enol, a phenol, or a combination thereof. In some embodiments, these acids are used as a compound with the disclosed isotopes (meaning the acids and the isotopes are bonded), or as a component (meaning that the acids and the isotopes are contained in the composition without forming bonds.) In some embodiments, the pharmaceutical composition includes, without limitation, in addition to including the enriched isotopes of chemical elements, lactic acid (present in breast milk), acetic acid (present in gastrointestinal tract and in lungs), formic acid (present in human skin fibroblasts), citric acid (present in bones and in blood), oxalic acid (present in kidneys), uric acid (present in liver), malic acid (present in certain vitamins), tartaric acid (present in fruits and vegetables), butyric acid (present in animal products), and more. In some embodiments, the carboxylic acids is added to the composition with the isotopes to enhance metabolic processes and anti-microbial activities of an organism.

In some embodiments, the pharmaceutical composition includes dicarboxylic acids, for regulating metabolic pathways, such as ω-Oxidation and β-Oxidation, and energy production through producing acetyl-CoA and succinyl-CoA, which enter the tricarboxylic acid (TCA) cycle, providing substrates for energy production and replenishment of TCA cycle intermediates. Depending on disease, dicarboxylic acids can be included to regulate lipid metabolism and oxidation of fatty acids. Dicarboxylic acids can also be included in the disclosed composition of matter to therapeutically treat metabolic diseases such as type 2 diabetes. For example, dodecanedioic acid has shown promise in normalizing plasma glucose levels in diabetic patients without affecting insulin levels. Depending on the therapeutic targets, dicarboxylic acids can also be included to attain antiketogenic effects, when necessary.

In some embodiments, the composition includes a sulfonic acid, including perfluorooctane sulfonic acid (PFOS) and other related compounds, to improve absorption and excretion, by targeting lipid metabolism, immune system activation, as well as intestinal and endocrine processes. In other embodiments, the composition includes sulfonic acids to target developmental and reproductive effects, respiratory processes, and/or skin irritation. For example, including perfluorooctane sulfonic acid with the cited ⁶⁴Zn isotope enriched to exceed 48.61% can be used for the development of pharmaceutical compositions to target the development of autism.

In some embodiments, the composition includes amino acids to enhance protein synthesis, metabolic processes, and/or neurotransmission. For example, the enriched isotopes of ²⁴Mg, ²⁵Mg, ⁶⁴Zn, ⁶⁶Zn, ⁶³Cu, and/or ⁵⁴Fe, can be combined with L-aspartic acid to improve neurotransmission and mitochondrial function. L-leucine can be included with the isotopes for protein synthesis and muscle repair, and/or to regulate blood sugar levels, stimulate wound healing, and produce growth hormones. L-valine can be included with the enriched isotopes to stimulate muscle growth, tissue regeneration, and cellular energy production. L-histidine can be used in the composition with the enriched isotopes to develop pharmaceutical compositions to target digestion, sleep-wake cycles, and sexual function.

In some embodiments, the disclosed pharmaceutical composition includes unsaturated fatty acids to improve brain and eye health, target cardiovascular conditions, and regulation of metabolism. For example, including omega-3 fatty acids (alpha-linolenic acid, eicosapentaenoic acid, or docosahexaenoic acid) with ⁶⁴Zn isotope can target inflammation and improve immune function. More specifically, using docosahexaenoic acid with ⁵⁴Fe and/or ⁶⁴Zn and/or ⁸⁵Rb isotopes can target improvement of overall brain health and cognitive function. Using unsaturated fatty acids with the lightest isotopes is of particular importance because they feature stronger bonds between carbon atoms, while the lightest isotopes feature weaker covalent bonds. This means that less cellular energy is required to break the weaker covalent bonds of the lightest isotopes (hence less stress on mitochondria organelles.)

In some embodiments, the pharmaceutical composition includes aromatic acids to improve antioxidant and anti-inflammatory activities of an organism, as well as to enhance cardiovascular and digestive health. For example, salicylic acid can be included with ⁵⁴Fe, ²⁴Mg, ⁶⁴Zn, and/or ⁴⁰Ca to target treatment of acne and other skin conditions through the improvement of anti-inflammatory and analgesic bioactivities. Cinammic acid can be included with ⁶⁴Zn, ⁶³Cu, ⁷⁴Se and/or ²⁴Mg to target antimicrobial in addition to antioxidant properties. However, the disclosed composition must not include polycyclic aromatic hydrocarbons (PAHs); these must be specifically excluded due to their toxic effects.

In some embodiments, the pharmaceutical composition includes hydroxy acids, depending on the therapy targets, to enhance the exfoliation of dead skin cells and skin renewal, hydration, and collagen synthesis. In some embodiments, the pharmaceutical composition includes hydroxy acids to enhance anti-inflammatory and antioxidant activities or/and inhibition of tyrosinase activity, when and as intended by the therapeutic goals. Furthermore, In some embodiments, the pharmaceutical composition includes hydroxy acids for modulation of matrix degradation and/or for photoprotection and photocarcinogenesis, including applications in cosmetics.

In some embodiments, the pharmaceutical composition includes thiols to enhance the redox reactions and antioxidant activity, redox signaling, enzyme catalysis, protein folding and structure. Enols and phenols can be included to stimulate anti-microbial, antioxidant, and anti-inflammatory activities of an organism.

The inclusion of these acids must be made selectively and taking into consideration the target pathology and other factors. Furthermore, alcohols and polycyclic aromatic hydrocarbons (PAHs) must be specifically excluded from the disclosed composition due to their toxic effects.

In some embodiments, the pharmaceutical composition includes ribonucleic acid (RNA) to improve protein synthesis and/or gene regulation. Examples include using RNA with ⁶⁴Zn to target improving and supporting memory, aiding in recovery from surgery or injury, and promoting digestive health.

In some embodiments, the at least one of a carboxylic acid, a dicarboxylic acid, a sulfonic acid, a hydroxy acid, an amino acid, a fatty acid, an aromatic acid, a thiol, an enol, a phenol, a ribonucleic acid, or a combination thereof can also be used as part of an enzyme, a peptide, or a protein. In some embodiments, the pharmaceutical composition further includes (in addition to the isotopes) at least one of an enzyme, a peptide, a protein, an oligonucleotide, nucleotide, an antibody, or a combination thereof. Notwithstanding, an enzyme, a peptide, a protein, an oligonucleotide, nucleotide, an antibody can be included in the disclosed composition as ready chain of one or more of the cited acids. For example, enzymes are composed of one or more polypeptide chains of amino acids, which fold into a specific three-dimensional structure. This structure includes an active site where the substrate—a molecule upon which the enzyme acts—binds. In some embodiments, the composition further includes at least one of an enzyme, a peptide, a protein, an oligonucleotide, a nucleotide, an antibody, or a combination thereof. In further embodiments, the composition includes the entire aggregate (ready) chains of amino acids in form of an enzyme. For avoidance of doubt, this rationale applies not only to enzymes, but also to peptides, proteins, oligonucleotides, nucleotides, and antibodies.

In some embodiments, the composition further includes at least one of a metal-ion binder, a protein nanocage, a chelating agent, a solute carrier, a metalloenzyme inhibitor, a microsphere, a polymeric micelle, a liposome, a hybrid nanoparticle, a nanoparticle, a membrane-derived vesicle, a nanosome, a noisome, an adeno-associated virus, a metal conjugator, or a combination thereof. The inclusion of these play functional role in the novel composition of matter, meaning that the cited carriers are functional to the isotopes and the acids (non-mutually inclusive) delivery to the target depending on the type of pathology, therapeutic goals, toxicity profile, and other considerations. For avoidance of doubt, the isotopes and the acids can be delivered as components of the same composition of matter, or separately.

In some embodiments, a liposome can be used to deliver an acid into ovarian cancer cells, while ⁶⁴Zn isotope is delivered to the same target using a nanocage. In this example, the dual impact can result in an eradication of the cancer cell colony. Notwithstanding the above, the same combination of ⁶⁴Zn isotope and L-aspartic amino acid can be delivered to the cells of ovarian cancer by including both in a stimuli-responsive liposomes (or an alternative type of liposomes), which can maintain stability in normal physiological conditions but release their payload specifically in the tumor region, improving targeting and minimizing off-target effects. Delivering zinc isotopes in atomic form to ovarian cancer cells using nanocages is another feasible example of application and could be an effective approach for targeted cancer therapy.

In certain embodiments, the pharmaceutical composition is administered in combination with a chelating agent. While a carboxylic acid, a dicarboxylic acid, a sulfonic acid, a hydroxy acid, an amino acid, a fatty acid, an aromatic acid, a thiol, an enol, a phenol, or a ribonucleic acid can act as chelating agents, the composition, in some embodiments, includes other types of chelating agents. These chelating agents include dimercaprol, deferoxamine, deferasirox, deferiprone, trientine, and penicillamine. An example of this chelating agents, dimercaprol, is a British anti-Lewisite (BAL) that is used in oil, primarily, for heavy metal poisoning, which is accompanied by high level of inflammation and oxidative stress. The oxidative stress induced by heavy metals is one of known prime mechanisms behind their toxicity and ability to cause neurological disorders, cardiovascular diseases, cancer, etc. At the same time, these neurological disorders, cardiovascular diseases, and oncological pathologies can feature depletion of essential elements from the tissues. Specifically, for example, the brain of Alzheimer's disease patients can feature isotopic deficiency of ⁶⁴Zn and ⁸⁵Rb. ⁶⁴Zn has natural isotopic ratio of 48.60% in natural zinc samples. The disclosed composition has higher ratio (thus enriched) of ⁶⁴Zn. The bioavailability of natural zinc from blood to brain is relatively low due to the regulatory function of the blood-brain barrier (BBB). Some studies suggest that only 2-3% of zinc in the blood is taken up by brain tissues. Hence, only <1.485% of ⁶⁴Zn can result in cellular uptake in the brain. Since liposomes are predominantly internalized by energy-dependent endocytic pathways, the ⁶⁴Zn can be delivered to brain cells using liposomes. In this example, the atomic form of ⁶⁴Zn combined with BAL can be delivered to brain cells with a liposome without making valent bonds. This could target the dual action of relieving the brain cells from heavy metal toxicity while supplying the micronutrient ⁶⁴Zn required for several cellular processes (for example healthy mitochondrial function.)

In another aspect, this disclosure provides a method of modulating local and/or systemic inflammation in mammals including administering a therapeutically effective amount to a subject in need thereof of at least one isotope-specific chemical element selected from ⁶Li, ¹⁰B, ²⁴Mg, ²⁵Mg, ²⁸Si, ²⁹Si, ³²S, ³³S, ³⁹K, 35Cl, ⁴⁰Ca, ⁴²Ca, ⁴³Ca, ⁵⁰V, ⁵⁰Cr, ⁵²Cr, ⁵⁴Fe, ⁵⁶Fe, ⁵⁸Ni, ⁶⁰Ni, ⁶¹Ni, ⁶³Cu, ⁶⁴Zn, ⁶⁶Zn, ⁸⁴Sr, ⁹²Mo, ⁹⁴Mo, ⁹⁵Mo, ⁹⁶Mo, ⁷⁴Se, ⁷⁶Se, ⁷⁷Se, ⁷⁸Se, ⁷⁰Ge, ⁷²Ge, ⁷³Ge, ⁶⁹Ga, ⁷⁹Br, ⁸⁵Rb, ¹⁰⁷Ag, ¹³⁰Ba, ¹³²Ba, ¹³⁴Ba, ¹³⁵Ba, and/or ¹³⁶Ba present in elemental form, atomic form, colloidal substance form, in form of pharmaceutically acceptable compound, or in form of salt, or in combination of these forms, wherein ⁶Li is enriched to exceed 10.06%, 12C is enriched to exceed 98.93%, ¹⁰B is enriched to exceed 21.01%, ²⁴Mg is enriched to exceed 80.01%, ²⁵Mg is enriched to exceed 10.01%, ²⁸Si is enriched to exceed 92.51%, ²⁹Si is enriched to exceed 5.01%, ³²S is enriched to exceed 95.03%, ³³S is enriched to exceed 0.91%, ³⁹K is enriched to exceed 93.51%, ³⁵Cl is enriched to exceed 76.01%, ⁴⁰Ca is enriched to exceed 96.95%, ⁴²Ca is enriched to exceed 0.65%, ⁴³Ca is enriched to exceed 0.65%, ⁵⁰V is enriched to exceed 0.25%, ⁵⁰Cr is enriched to exceed 4.25%, ⁵²Cr is enriched to exceed 85.01%, ⁵⁴Fe is enriched to exceed 5.85%, ⁵⁶Fe is enriched to exceed 91.8%, ⁵⁸Ni is enriched to exceed 68.10%, ⁶⁰Ni is enriched to exceed 26.51%, ⁶¹Ni is enriched to exceed 1.12%, ⁶³Cu is enriched to exceed 69.10%, ⁶⁴Zn is enriched to exceed 58.61%, ⁶⁶Zn is enriched to exceed 28.10%, ⁸⁴Sr is enriched to exceed 0.60%, ⁹²Mo is enriched to exceed 15.01%, ⁹⁴Mo is enriched to exceed 9.25%, ⁹⁵Mo is enriched to exceed 15.95%, ⁹⁶Mo is enriched to exceed 16.69%, ⁷⁴Se is enriched to exceed 0.90%, ⁷⁶Se is enriched to exceed 9.25%, ⁷⁷Se is enriched to exceed 7.65%, ⁷⁸Se is enriched to exceed 23.81%, ⁷⁰Ge is enriched to exceed 20.55%, ⁷²Ge is enriched to exceed 27.57%, ⁷³Ge is enriched to exceed 7.77%, ⁶⁹Ga is enriched to exceed 64.11%, ⁷⁹Br is enriched to exceed 50.70%, ⁸⁵Rb is enriched to exceed 72.20%, ¹⁰⁷Ag is enriched to exceed 51.61%, ¹³⁰Ba is enriched to exceed 0.12%, ¹³²Ba is enriched to exceed 0.12%, ¹³⁴Ba is enriched to exceed 2.42%, ¹³⁵Ba is enriched to exceed 6.60%, and/or ¹³⁶Ba is enriched to exceed 7.86%. The isotopes can be administered in atomic form, elemental form, colloidal substance form, or in form of pharmaceutically acceptable compound or salt, or as a combination of these forms, as applicable.

In another aspect, this disclosure provides a method of modulating local and/or systemic inflammation in mammals including administering a therapeutically effective amount to a subject in need thereof a disclosed pharmaceutical composition.

The terms “elemental form” and “atomic form” refer to different aspects of the same chemical elements and their structures. The elemental form can exist as mono-isotopic, di-isotopic, or poly-isotopic molecules. The atomic form exists as the isolated atoms (isotopes) of an element, which are the building blocks of matter and/or molecules. The chemical behavior of the two is influenced by how atoms (isotopes) are bonded together in molecules and other factors. Understanding these distinctions is crucial for studying the properties and behaviors of the isotopes in various biological functions, such as inflammation and oxidative stress in this case.

The term “colloidal substance form” refers to a type of mixture where one or more substances are dispersed evenly throughout a medium (carrier) substance. As used herein, the isotopic particles in a colloid are larger than those in a true solution but smaller than those in a suspension, typically ranging from 1 nanometer to 1 micrometer in size. Colloids are heterogeneous mixtures, meaning the dispersed particles are not uniformly distributed at the molecular level but are evenly dispersed throughout the continuous phase. Colloidal particles do not settle out of the mixture upon standing and cannot be separated by ordinary filtration methods. However, they can be separated by centrifugation.

In some embodiments, the isotopes in the disclosed composition is dispersed in sanitized water, distilled water, demineralized water, ionized water, deuterium-depleted water, pharmaceutically acceptable oil, natural polymer, synthetic polymer, carboxymethylcellulose, methylcellulose, hydroxypropyl cellulose, and/or a combination thereof. For avoidance of doubt, the cited isotopes can be dispersed and administered in elemental or atomic form, individually or as a combination of two or more cited isotopes, or as a component of a pharmaceutical compound. In some embodiments, the water forms is made with the above-described cellulose derivatives to increase viscosity and stability of the suspensions, and/or with natural polymers to improve microbial contamination or synthetic polymers to improve viscosity. A pharmaceutically acceptable oil can also be a suspension agent, for example omega-3 fish oil (C₂₂H₃₂O₂.) The term “pharmaceutically acceptable compound” refers to a substance that is suitable for use in pharmaceutical formulations due to its safety, efficacy, and toxicity profile. In some embodiments, the disclosed composition includes one or more pharmaceutically acceptable compounds including at least one non-steroidal anti-inflammatory drug selected from ibuprofen, aspirin, naproxen, diclofenac, etodolac, fenoprofen, flurbiprofen, indomethacin, meclofenamate, mefenamic acid, nabumetone, naproxen, oxaprozin, piroxicam, sulindac, tolmetin, a COX-2 inhibitor, or a combination thereof provides that at least one of non-steroidal anti-inflammatory drug selected from ibuprofen, aspirin, naproxen, diclofenac, etodolac, fenoprofen, flurbiprofen, indomethacin, meclofenamate, mefenamic acid, nabumetone, naproxen, oxaprozin, piroxicam, sulindac, tolmetin, COX-inhibitor, or a combination thereof, can be included with the pharmaceutically acceptable compound.

In some embodiments, the pharmaceutically acceptable compound includes at least one of an enzyme inhibitor, an ATP-binding agent, a solute-linked carrier, an organic anion or cation, a non-organic anion or cation, a polymeric nanocarrier, a metal-organic framework, a lipid-based nanoparticle, a dendrimer, a nanostructured lipid carrier, a carbon nanotube, or a combination thereof. These are carriers used for targeted drug delivery.

In some embodiments, the disclosed composition includes isotopes in form of salt, which can be an organic salt, an inorganic salt, a chelating agent, or a combination thereof; in some embodiments, the disclosed method includes administering isotopes in form of salt, which can be an organic salt, an inorganic salt, a chelating agent, or a combination thereof.

The term “salt” refers to a salt form that maintains the biological effectiveness and properties of the co-included cited isotope, whether in elemental or in atomic form, while remaining safe, non-toxic, and suitable for use in pharmaceutical formulations. These salts are used to improve solubility, stability, absorption, excretion, and manufacturability of the active pharmaceutical ingredient (API)—one or more of the cited isotopes in this case. Furthermore, these salts are designed to be non-toxic and biologically effective. The choice of a particular salt form is based on various factors, including the chemistry of the API, the intended dosage form, pharmacokinetics, and pharmacodynamics. The goal here is to achieve the best possible therapeutic and pharmaceutical profile.

In another aspect, this disclosure provides a method of reducing oxidative stress including administering a therapeutically effective amount to a subject in need thereof of at least one isotope-specific chemical element selected from 6Li, 12C, 10B, 24Mg, 25Mg, 28Si, 29Si, 32S, 33S, 39K, 35Cl, 40Ca, 42Ca, 43Ca, 50V, 50Cr, 52Cr, 54Fe, 56Fe, 58Ni, 60Ni, 61Ni, 63Cu, 64Zn, 66Zn, 84Sr, 92Mo, 94Mo, 95Mo, 96Mo, 74Se, 76Se, 77Se, 78Se, 70Ge, 72Ge, 73Ge, 69Ga, 79Br, 85Rb, 107Ag, 130Ba, 132Ba, 134Ba, 135Ba, and/or 136Ba, present in elemental form, atomic form, colloidal substance form, in form of pharmaceutically acceptable compound, or in form of a salt, wherein 6Li is enriched to exceed 10.06%, 12C is enriched to exceed 98.93%, 10B is enriched to exceed 21.01%, 24Mg is enriched to exceed 80.01%, 25Mg is enriched to exceed 10.01%, 28Si is enriched to exceed 92.51%, 29Si is enriched to exceed 5.01%, 32S is enriched to exceed 95.03%, 33S is enriched to exceed 0.91%, 39K is enriched to exceed 93.51%, 35Cl is enriched to exceed 76.01%, 40Ca is enriched to exceed 96.95%, 42Ca is enriched to exceed 0.65%, 43Ca is enriched to exceed 0.65%, 50V is enriched to exceed 0.25%, 50Cr is enriched to exceed 4.25%, 52Cr is enriched to exceed 85.01%, 54Fe is enriched to exceed 5.85%, 56Fe is enriched to exceed 91.8%, 58Ni is enriched to exceed 68.10%, 60Ni is enriched to exceed 26.51%, 61Ni is enriched to exceed 1.12%, 63Cu is enriched to exceed 69.10%, 64Zn is enriched to exceed 58.61%, 66Zn is enriched to exceed 28.10%, 84Sr is enriched to exceed 0.60%, 92Mo is enriched to exceed 15.01%, 94Mo is enriched to exceed 9.25%, 95Mo is enriched to exceed 15.95%, 96Mo is enriched to exceed 16.69%, 74Se is enriched to exceed 0.90%, 76Se is enriched to exceed 9.25%, 77Se is enriched to exceed 7.65%, 78Se is enriched to exceed 23.81%, 70Ge is enriched to exceed 20.55%, 72Ge is enriched to exceed 27.57%, 73Ge is enriched to exceed 7.77%, 69Ga is enriched to exceed 64.11%, 79Br is enriched to exceed 50.70%, 85Rb is enriched to exceed 72.20%, 107Ag is enriched to exceed 51.61%, 130Ba is enriched to exceed 0.12%, 132Ba is enriched to exceed 0.12%, 134Ba is enriched to exceed 2.42%, 135Ba is enriched to exceed 6.60%, and/or 136Ba is enriched to exceed 7.86%. The isotopes must be enriched to the stated ratios and can be present in elemental form, atomic form, colloidal substance form, in form of pharmaceutically acceptable compound, or in form of a salt, or in a combination of these forms, as applicable.

In another aspect, this disclosure provides a method of reducing oxidative stress including administering a therapeutically effective amount to a subject in need thereof a disclosed pharmaceutical composition. In some embodiments, the colloidal form further includes at least one of ozonized water, distilled water, demineralized water, ionized water, and/or deuterium-depleted water, or a combination thereof. For example, deuterium-depleted water can be used with or without natural polymers to improve microbial contamination or synthetic polymers to improve viscosity. This form of water alone has been shown to enhance the activity of intrinsic antioxidant enzymes such as superoxide dismutases (SOD1 and SOD2) and catalases, which help neutralize reactive oxygen species and mitigate oxidative stress, which is important for developing neuroprotective therapies. Furthering this example, dispersing ⁶⁴Zn, ⁷⁴Se, ⁶³Cu, or ²⁴Mg isotopes in deuterium-depleted water and administering the resulting substance to the target subject can result in a decrease of oxidative stress through various mechanisms, including acting as cofactors for antioxidant enzymes and participating in redox reactions. For avoidance of doubt, the cited isotopes can be dispersed and administered in elemental or atomic form, individually or as a combination of two or more cited isotopes, or as a component of a pharmaceutical compound. These water forms can contain cellulose derivatives to increase viscosity and stability of the suspensions. A pharmaceutically acceptable oil (for example omega-3 fish oil, or C₂₂H₃₂O₂) can also be used for fat-soluble isotopes.

In some embodiments, the method further includes administering at least one of paracetamol, resveratrol, beta-carotene, vitamin B, vitamin C, vitamin D, vitamin E, glutathione, coenzyme Q10, caffein, and/or or a combination thereof, the inclusion of which can enhance the antioxidant defense. For example, resveratrol, a common dietary supplement, acts as an antioxidant, protecting cells from oxidative damage. It has also shown anti-inflammatory, anticancer, cardioprotective, and neuroprotective properties. Combining resveratrol with ⁶⁴Zn, ⁷⁴Se, ⁶³Cu, or ²⁴Mg and other isotopes can enhance its known therapeutic activities and allow development of target medicines for several diseases.

In some embodiments, the method further includes administering at least one of a proteinogenic amino acid or nonessential amino acid or a combination thereof.

In some embodiments, the method further includes a product of at least one of a proteinogenic amino acid or nonessential amino acid or a combination thereof.

This disclosure provides a safer and more efficient composition of matter and methods of reducing local and systemic inflammation and oxidative stress. This goal is achieved by using stable (non-radioactive) isotopes of essential chemical elements, which have been historically used in their natural isotopic state.

As used herein, the word “a” or “plurality” before a noun represents one or more of the particular noun. For the terms “for example” and “such as,” and grammatical equivalences thereof, the phrase “and without limitation” is understood to follow unless explicitly stated otherwise. As used herein, the term “about” is meant to account for variations due to experimental error. All measurements reported herein are understood to be modified by the term “about,” whether or not the term is explicitly used, unless explicitly stated otherwise. As used herein, the singular forms “a,” “an,” and “the” include plural referents unless the context clearly dictates otherwise.

All ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less, e.g., 1.0 to 5.3, or 4.7 to 10.0, or 3.6 to 7.9. All ranges disclosed herein are also to be considered to include the end points of the range, unless expressly stated otherwise. For example, a range of “between 5 and 10” or “5 to 10” or “5-10” should be considered to include the end points 5 and 10.

It is further to be understood that the feature or features of one embodiment can generally be applied to other embodiments, even though not specifically described or illustrated in such other embodiments, unless expressly prohibited by this disclosure or the nature of the relevant embodiments. Likewise, compositions and methods described herein can include any combination of features and/or steps described herein not inconsistent with the objectives of the present disclosure. Numerous modifications and/or adaptations of the compositions and methods described herein will be readily apparent to those skilled in the art without departing from the present subject matter.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The term “isotope”, as used herein, refers to a variant of an atom of a chemical element that defer by the number of neutrons in their nucleuses and have a different atomic mass. According to the proton-neutron model developed by D. I. Ivanenko and W. Heisenberg (1932), atoms of all chemical elements consist of three types of elementary particles: positively charged protons, negatively charged electrons, and neutrons that have no charge. The number of protons p in the nucleus determines the atomic number Z of the chemical element in Mendeleev's periodic table. The proton and the neutron, which have a common name—nucleons—have almost identical weight. The mass of the neutron (1.00866 amu) is somewhat greater than the proton mass (1.00727 amu). The electron mass is much smaller than that of the nucleons (for example, the proton-to-electron mass ratio is 1836.13). Therefore, the mass of the atom is concentrated in its nucleus. Hence, the mass number of the atom A is connected with the atomic number by a simple relation A=p+n=Z+n, where n is the number of neutrons in the nucleus of an atom. The number of protons in the nucleus of an atom uniquely determines the position of an element in the periodic table of the elements. Furthermore, the number of protons determines the number of electrons present in a neutral atom thus determining the chemical properties of this atom. However, atoms with the same atomic number Z (and hence the number of protons p) can have different neutron numbers n. Thus atoms with different atomic mass numbers can occupy the same position on the periodic table. Chemical elements having the same atomic number but a different atomic mass are known as isotopes.

Terms “natural abundance” of an isotope refers to the fraction of the total amount of the corresponding element that the isotope represents, on a mole-fraction basis (that is, not, for example, on a mass basis). For example, if ⁶⁴Zn has a natural abundance of 48.6%, that means that 48.6% of Zn atoms on earth are the ⁶⁴Zn atoms. Isotopes are atoms with different number of neutrons in their nuclei.

The term “relative isotopic ratio” refers to a measure of the proportion of one isotope of a chemical element relative to another isotope of the sample of the same element, typically expressed in comparison to a standard reference. For example, the isotopic ratio of ⁶⁴Zn atoms of naturally occurring zinc is 48.6%.

The term “enriched” refers to the process of increasing the proportional ratio of a specific isotope within a mixture of isotopes. The enrichment process exploits the differences in physical and/or chemical properties between isotopes of the same element to separate and concentrate one isotope from the others. When a composition is “enriched” for a certain isotope, the abundance of the isotope in the composition is greater than the isotope's natural abundance. For the preceding ⁶⁴Zn example, a composition in which ⁶⁴Zn constitutes more than 48.6% of the total Zn in the composition, on a mole-fraction basis, would be “enriched” for ⁶⁴Zn.

The term “amino acid” is known in the art and refers to biologically active components that are critical to the structure and function of all living cells. They are organic compounds composed of nitrogen, carbon, hydrogen, and oxygen, along with a variable side chain group. Each amino acid features a central carbon atom (C), known as the alpha (α) carbon, to which an amino group (NH2), a carboxyl group (COOH), a hydrogen atom (H), and a distinctive side chain (R group) are attached. The chemical nature of the side chain determines the physical and chemical properties of the amino acid, influencing how amino acids interact with each other and with other molecules. Amino acids are the monomers that link together in specific sequences to form proteins and enzymes, which perform a vast array of functions in the body, including catalyzing metabolic reactions, DNA replication, responding to stimuli, and transporting molecules from one location to another. Some amino acids serve as precursors for neurotransmitters. For example, tryptophan is a precursor for serotonin, and tyrosine is a precursor for dopamine and norepinephrine. Some other amino acids like cysteine play a role in the immune system by helping to produce antibodies. Examples of amino acids include but are not limited to alanine, arginine, aspartate, glutamine, histidine, nitrilotriacetic acid (NTA), and leucine.

The term “enzyme” is known in the art and refers to a biological catalyst that accelerates chemical reactions within living organisms without being consumed or permanently altered by the reaction. Enzymes function by lowering the activation energy required for a reaction to proceed, thereby increasing the reaction rate and allowing cellular processes to occur efficiently and rapidly under mild conditions. Each enzyme is specific to a particular reaction or type of reaction, a property derived from its unique three-dimensional structure. Examples of enzymes include but are not limited to digestive enzymes (pepsin, amylase, lactase, etc.), metabolic enzymes (creatine kinase, etc.), miscellaneous enzymes (catalase, thrombine, Lysozyme, etc.)

The term “peptide” refers to a short chain of amino acids linked by peptide bonds, which are formed through a dehydration synthesis reaction between the carboxyl group of one amino acid and the amino group of another. Peptides play various roles in the body, acting as hormones, neurotransmitters, growth factors, and antibiotics, among other functions. They are crucial for many biological processes, including cell signaling, immune responses, and metabolism. Examples of biologically active peptides include insulin, which regulates glucose levels in the blood; glucagon, which has the opposite effect of insulin; and oxytocin, which is involved in childbirth and emotional bonding. The term “peptide” as used in this present invention includes polypeptides.

The term “anion transporting polypeptide” refers to a membrane transport that facilitates the cellular uptake of a wide range of organic anions, including various endogenous substances like bile acids, steroid hormones, and thyroid hormones, as well as exogenous substances such as drugs and toxins. Organic anion transporting polypeptides are encoded by the SLCO gene family and are characterized by their ability to transport large and amphipathic molecules across cell membranes in an ATP-independent manner, often functioning as electroneutral exchangers or facilitated transporters. They are expressed in various tissues, including the liver, kidney, intestine, and brain, impacting the pharmacokinetics and pharmacodynamics of many drugs, which makes them significant in the context of biological interactions. One example of an anion transporting polypeptide is OATP1B1. This transporter is primarily located in the liver on the basolateral membrane of hepatocytes and plays a significant role in the hepatic uptake of a wide range of substrates from the blood into the liver cells, contributing to the metabolism and biliary excretion of various endogenous compounds such as bile acids, thyroid hormones, and steroid hormone conjugates, as well as exogenous substances including drugs like statins.

The term “protein” is known in the art and refers to a distinct class of biological molecules due to their ability to coagulate or flocculate under treatments with heat or acid. Proteins are essential for the structure, function, and regulation of the body's tissues and organs. As used herein, term “proteins” further refers to structural proteins, transport proteins, hormonal proteins, defense proteins, storage proteins, contractile proteins, receptor proteins, globular proteins, and fibrous proteins. Examples of structural proteins include collagen and elastin found in connective tissues and keratin found in hair and nails. Examples of transport proteins are hemoglobin, which transports oxygen through the blood, and membrane transport proteins like the GLUT4 transporter. Example of hormonal proteins is insulin, which regulates glucose metabolism by controlling the blood-sugar concentration. Example of defense proteins is immunoglobulin, which attacks and neutralizes pathogens such as bacteria and viruses. Casein in milk and ovalbumin in egg whites are examples of storage proteins that provide nutrients. Examples of contractile proteins include actin and myosin, which are involved in muscle contraction and movement. Keratin is an example of fibrous proteins.

The term “oligonucleotide” is known in the art and refers to a short nucleic acid chain, usually consisting of up to approximately 20 nucleotides. These sequences can be composed of DNA, RNA, or their analogs and are typically synthesized by polymerizing individual nucleotide precursors. Oligonucleotides are crucial in molecular biology and medicine for their ability to bind specifically to complementary nucleotide sequences, influencing gene expression and regulation. Examples include antisense oligonucleotides (ASOs), small interfering RNAs (siRNAs), microRNA (miRNA), molecular probes, aptamers, triplex-forming oligonucleotides (TFOs), locked nucleic acids (LNAs), morpholinos, spiegelmers, and PCR primers.

The term “antibody” is known in the art and refers to specialized Y-shaped immunoglobulins produced by the immune system to identify and neutralize foreign substances such as bacteria, viruses, fungi, and toxins. Antibodies play a crucial role in the body's defense mechanism by recognizing and binding to specific antigens, which are molecules on the surface of pathogens or foreign particles. Examples include immunoglobulins G (IgG), IgM, IgA, IgE, and IgD.

The term “metal-ion binder” refers to chemical components that work by binding to metal ions, forming stable, water-soluble complexes with metal ions through coordinate or covalent bonds. Examples include ethylenediaminetetraacetic acid (EDTA), dimercaprol (British Anti-Lewisite or BAL), deferoxamine, penicillamine, and succimer (DMSA).

The term “salt” refers to organic and inorganic salts are two broad categories of active compounds that play significant roles in various biological processes. Organic salts are characterized by the presence of carbon-hydrogen (C—H) bonds within their molecular structure. These salts typically result from the reaction of organic acids with bases. The cation (positively charged ion) in these salts often includes organic groups, which can significantly influence the properties and applications of the salts. Inorganic salts do not contain carbon-hydrogen bonds. They are typically formed by the reaction of inorganic acids with bases. The ions in inorganic salts can include metals or other elements from across the periodic table, leading to a vast array of compounds with diverse properties. The fundamental differences between organic and inorganic salts lie in their chemical structure and resultant physical properties. Organic salts, with their organic cations or anions, often participate in organic reactions and have specific uses in organic synthesis and pharmaceutical formulations. In contrast, inorganic salts, with their diverse range of cations and anions, are pivotal in processes that require high thermal stability and solubility in water. Examples of organic salts include but are not limited to sodium acetate (CH3COONa), potassium citrate (K3C6H5O7), magnesium stearate (C36H70MgO4), and benzalkonium chloride (C22H40ClN). Examples of inorganic salts include but are not limited to sodium chloride (NaCl), potassium sulfate (K2SO4), magnesium sulfate (MgSO4), and zinc oxide (ZnO).

The term “metalloenzyme inhibitor” refers to organic compounds that target metalloenzymes, which are enzymes that require metal ions to function properly. These inhibitors are significant in therapeutic applications, particularly in treating diseases where metalloenzymes play a crucial role. Metalloenzymes incorporate metal ions in their active sites, which are essential for their catalytic activity. These enzymes are involved in a wide range of biological processes, such as metabolism, DNA synthesis, and the regulation of gene expression. Common metallomic elements found in these enzymes include zinc, iron, copper, manganese, and others. Recent research has focused on improving the efficacy and selectivity of metalloenzyme inhibitors through advanced screening techniques and better understanding of metalloenzyme biology. Examples of metalloenzyme inhibitors include but are not limited to carbonic anhydrase inhibitors, matrix metalloproteinases (MMPs) inhibitors, angiotensin-converting enzyme (ACE) inhibitors, histone deacetylase (HDAC) inhibitors, and zinc-containing metalloenzymes inhibitors.

The term “microspheres” refers to spherical particles ranging in size from 1 to 1000 micrometers and used for drug delivery. These particles can be made from various materials, including natural and synthetic polymers, and are designed to encapsulate drugs, providing controlled and sustained release. The use of microspheres in pharmaceuticals offers several advantages, including improved drug stability, targeted delivery, and enhanced patient compliance. Examples include biodegradable microspheres made from the materials such as polylactic acid (PLA), polyglycolic acid (PGA), and their copolymers (PLGA); non-biodegradable microspheres, magnetic microspheres, and floating microspheres.

The term “polymeric micelle” refers to nanoscale colloidal carriers formed by the self-assembly of amphiphilic block copolymers in aqueous solutions. Polymeric micelles have emerged as a significant tool in the field of drug delivery, particularly for the treatment of cancer, due to their unique core-shell structure that enables them to solubilize hydrophobic drugs, enhance drug stability, and facilitate targeted delivery. Examples include NK105 usually incapsulating chemotherapy drugs (i.e. Paclitaxel), NK012 formed from PEG-b-poly(1-glutamic acid) and containing Irinotecan; and NK911 for carrying Doxorubicin-loaded polymeric micelles using PEG-b-poly (α,β-aspartic acid).

The term “liposomes” refers to spherical vesicles composed of one or more phospholipid bilayers, which can encapsulate both hydrophilic and hydrophobic drugs. Examples include small unilamellar vesicles (SUVs), large unilamellar vesicles (LUVs), and multilamellar vesicles (MLVs).

The term “nanosomes” refers to nanocarriers used in precision nanomedicine to deliver therapeutic drugs to specific cells or tissues. Nanosomes have a unique structure consisting of a liposomal bilayer around a hydrophilic core, which can encapsulate either therapeutic drugs or functional biomolecules. This structure allows them to pass through biological barriers and target specific cells or tissues, thereby reducing the side effects associated with traditional drug delivery systems. Examples include nanosome minoxidil, nanosomes carrying Doxorubicin, and nanosome-based topical treatments.

The term “niosome” refers to is a type of vesicle used in drug delivery systems, composed primarily of non-ionic surfactants and cholesterol. These vesicles are microscopic and lamellar, meaning they have a layered structure similar to liposomes but are generally more stable and less expensive to produce. Examples include transferrin-conjugated pluronic niosomes, folic acid-functionalized niosomes, and chitosan-adorned niosomes.

The term “adeno-associated virus” refers to (AAVs) small, non-enveloped viruses that belong to the Parvoviridae family and the Dependoparvovirus genus. They have gained significant attention in the field of gene therapy due to their unique properties, including low immunogenicity, the ability to infect both dividing and non-dividing cells, and the capacity for long-term gene expression without integrating into the host genome. Examples include voretigene neparvovec-rzyl, onasemnogene abeparvovec-xioi, and alipogene tiparvovec.

The term “metal conjugate” refers to conjugation of metal-based compounds with drugs, polymers, or other carriers to improve drug delivery and therapeutic outcomes. Examples include metal nanoparticles (MNPs), metal-organic frameworks (MOFs), metallopolymers, and metal-based antibody drug conjugates (ADCs).

The term “inflammation” refers to a complex biological response of the body's immune system to harmful stimuli, such as pathogens, damaged cells, or irritants. The inflammation differs into local and systemic inflammation.

The term “oxidative stress” refers to a condition characterized by an imbalance between the production of reactive oxygen species (ROS), commonly known as free radicals, and the body's ability to detoxify these reactive intermediates or repair the resulting damage. This imbalance can lead to cellular and tissue damage and is implicated in various diseases and aging processes.

The term “metallome” refers to all metal and metalloid-containing molecules in a biological system and their distribution, isotopes (atoms), and chemical forms (species) of metal ions and inorganic elements bound to proteins, enzymes, nucleic acids, and other biomolecules.

The term “non-metallome” refers to all non-metal and non-metalloid-containing molecules in a biological system and their distribution, isotopes (atoms), and chemical forms (species) of metal ions and inorganic elements bound to proteins, enzymes, nucleic acids, and other biomolecules.

For the purpose of clarity, the disclosed compositions and methods include isotopes of metallome and non-metallome elements.

The “natural abundance” of an isotope refers to the fraction of the total amount of the corresponding element that the isotope represents, on a mole-fraction basis (that is, not, for example, on a mass basis). For example, if ⁶⁴Zn had an earth natural abundance of 48.63%, that would mean that 48.63% of Zn atoms on earth are the isotope ⁶⁴Zn. When a composition is “enriched” for a certain isotope, the abundance of the isotope in the composition is greater than the isotope's natural abundance. For the preceding ⁶⁴Zn example, a composition in which ⁶⁴Zn constitutes more than 48.63% of the total Zn in the composition, on a mole-fraction basis, would be “enriched” for ⁶⁴Zn.

“Effective amount,” “prophylactically effective amount,” or “therapeutically effective amount” refers to an amount of an agent or composition that provides a beneficial effect or favorable result to a subject, or alternatively, an amount of an agent or composition that exhibits the desired in vivo or in vitro activity. “Effective amount,” “prophylactically effective amount,” or “therapeutically effective amount” refers to an amount of an agent or composition that provides the desired biological, therapeutic, and/or prophylactic result. That result can be reduction, amelioration, palliation, lessening, delaying, and/or alleviation of one or more of the signs, symptoms, or causes of a disease, disorder or condition in a patient/subject, or any other desired alteration of a biological system. An effective amount can be administered in one or more administrations.

An “effective amount,” “prophylactically effective amount,” or “therapeutically effective amount” can be first estimated either in accordance with cell culture assays or using animal models, typically mice, rats, guinea pigs, rabbits, dogs or pigs. An animal model can be used to determine an appropriate concentration range and route of administration. Such information can then be used to determine appropriate doses and routes of administration for humans. When calculating a human equivalent dose, a conversion table such as that provided in Guidance for Industry: Estimating the Maximum Safe Starting Dose in Initial Clinical Trials for Therapeutics in Adult Healthy Volunteers (U.S. Department of Health and Human Services, Food and Drug Administration, Center for Drug Evaluation and Research (CDER), July 2005) can be used. The person of ordinary skill in the art is aware of additional guidance that can also be used to develop human therapeutic dosages based on non-human data. An effective dose is generally 0.01 mg/kg to 2000 mg/kg of an active agent, preferably 0.05 mg/kg to 500 mg/kg of an active agent. An exact effective dose will depend on the severity of the disease, patient's general state of health, age, body weight and sex, nutrition, time and frequency of administration, combination(s) of medicines, response sensitivity and tolerance/response to administration and other factors that will be taken into account by a person skilled in the art when determining the dosage and route of administration for a particular patient based on his/her knowledge of the art. Such dose can be determined by conducting routine experiments and at the physician's discretion. Effective doses will also vary depending on the possibility of their combined use with other therapeutic procedures, such as the use of other agents.

As used herein, a “patient” and a “subject” are interchangeable terms and can refer to a human patient/subject, a dog, a cat, a non-human primate, a non-human mammal.

Light isotopes can be purchased.

The disclosed composition may be administered to a subject in need thereof by any suitable mode of administration, any suitable frequency, and at any suitable, effective dosage. The disclosed composition may be in any suitable form and may be formulated for any suitable means of delivery.

The disclosed composition can be co-administered with another appropriate agent or therapy.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the appended claims. Thus, while only certain features of the invention have been illustrated and described, many modifications and changes will occur to those skilled in the art. It is therefore to be understood that the appended claims are intended to cover all such modifications and changes as fall within the true spirit of the invention.

What is claimed is:

1. A pharmaceutical composition for modulating inflammation and oxidative stress in mammals including at least one chemical element selected from H, Li, C, B, N, O, Mg, Si, S, K, Cl, Ca, V, Cr, Fe, Ni, Cu, Zn, Sr, Mo, Se, Ge, Ga, Br, Rb, Ag, and Ba, or a combination thereof, wherein said chemical elements are altered from their natural sample state so that ¹H is enriched to exceed 99.985%, ⁶Li is enriched to exceed 10.06%, ¹²C is enriched to exceed 98.93%, ¹⁰B is enriched to exceed 21.01%, ¹⁴N is enriched to exceed 99.64%, ¹⁶O is enriched to exceed 99.81%, ¹⁷O is enriched to exceed 0.01%, ²⁴Mg is enriched to exceed 80.01%, ²⁵Mg is enriched to exceed 10.01%, ²⁸Si is enriched to exceed 92.51%, ²⁹Si is enriched to exceed 5.01%, ³²S is enriched to exceed 95.03%, ³³S is enriched to exceed 0.91%, ³⁹K is enriched to exceed 93.51%, ³⁵Cl is enriched to exceed 76.01%, ⁴⁰Ca is enriched to exceed 96.95%, ⁴²Ca is enriched to exceed 0.65%, ⁴³Ca is enriched to exceed 0.65%, ⁵⁰V is enriched to exceed 0.25%, ⁵⁰Cr is enriched to exceed 4.25%, ⁵²Cr is enriched to exceed 85.01%, ⁵⁴Fe is enriched to exceed 5.85%, ⁵⁶Fe is enriched to exceed 91.8%, ⁵⁸Ni is enriched to exceed 68.10%, ⁶⁰Ni is enriched to exceed 26.51%, ⁶¹Ni is enriched to exceed 1.12%, ⁶³Cu is enriched to exceed 69.10%, ⁶⁴Zn is enriched to exceed 58.61%, ⁶⁶Zn is enriched to exceed 28.10%, ⁸⁴Sr is enriched to exceed 0.60%, ⁹²Mo is enriched to exceed 15.01%, ⁹⁴Mo is enriched to exceed 9.25%, ⁹⁵Mo is enriched to exceed 15.95%, ⁹⁶Mo is enriched to exceed 16.69%, ⁷⁴Se is enriched to exceed 0.90%, ⁷⁶Se is enriched to exceed 9.25%, ⁷⁷Se is enriched to exceed 7.65%, ⁷⁸Se is enriched to exceed 23.81%, ⁷⁰Ge is enriched to exceed 20.55%, ⁷²Ge is enriched to exceed 27.57%, ⁷³Ge is enriched to exceed 7.77%, ⁶⁹Ga is enriched to exceed 64.11%, ⁷⁹Br is enriched to exceed 50.70%, ⁸⁵Rb is enriched to exceed 72.20%, ¹⁰⁷Ag is enriched to exceed 51.61%, ¹³⁰Ba is enriched to exceed 0.12%, ¹³²Ba is enriched to exceed 0.12%, ¹³⁴Ba is enriched to exceed 2.42%, ¹³⁵Ba is enriched to exceed 6.60%, and/or ¹³⁶Ba is enriched to exceed 7.86%.

2. The pharmaceutical composition of claim 1, further including at least one of a carboxylic acid, a dicarboxylic acid, a sulfonic acid, a hydroxy acid, an amino acid, a fatty acid, an aromatic acid, a thiol, an enol, a phenol, a ribonucleic acid, or a combination thereof.

3. The pharmaceutical composition of claim, 1 further including at least one of an enzyme, a peptide, a protein, an oligonucleotide, a nucleotide, an antibody, or a combination thereof.

4. The pharmaceutical composition of claim 1, further including at least one of a metal-ion binder, a protein nanocage, a chelating agent, a solute carrier, an enzyme inhibitor, a microsphere, a polymeric micelle, a liposome, a hybrid nanoparticle, a nanoparticle, a membrane-derived vesicle, a nanosome, a noisome, an adeno-associated virus, a metal conjugator, or a combination thereof.

5. A method of modulating local and/or systemic inflammation in mammals including administering a therapeutically effective amount to a subject in need thereof of at least one isotope-specific chemical element selected from ⁶Li, ¹⁰B, ²⁴Mg, ²⁵Mg, ²⁸Si, ²⁹Si, ³²S, ³³S, ³⁹K, ³⁵Cl, ⁴⁰Ca, ⁴²Ca, ⁴³Ca, ⁵⁰V, ⁵⁰Cr, ⁵²Cr, ⁵⁴Fe, ⁵⁶Fe, ⁵⁸Ni, ⁶⁰Ni, ⁶¹Ni, ⁶³Cu, ⁶⁴Zn, ⁶⁶Zn, ⁸⁴Sr, ⁹²Mo, ⁹⁴Mo, ⁹⁵Mo, ⁹% Mo, ⁷⁴Se, ⁷⁶Se, ⁷⁷Se, ⁷⁸Se, ⁷⁰Ge, ⁷²Ge, ⁷³Ge, ⁶⁹Ga, ⁷⁹Br, ⁸⁵Rb, ¹⁰⁷Ag, ¹³⁰Ba, ¹³²Ba, ¹³⁴Ba, ¹³⁵Ba, and/or ¹³⁶Ba present in elemental form, atomic form, colloidal substance form, in form of pharmaceutically acceptable compound, or in form of salt, or in combination of these forms, wherein ⁶Li is enriched to exceed 10.06%, ¹²C is enriched to exceed 98.93%, ¹⁰B is enriched to exceed 21.01%, ²⁴Mg is enriched to exceed 80.01%, ²⁵Mg is enriched to exceed 10.01%, ²⁸Si is enriched to exceed 92.51%, ²⁹Si is enriched to exceed 5.01%, ³²S is enriched to exceed 95.03%, ³³S is enriched to exceed 0.91%, ³⁹K is enriched to exceed 93.51%, ³⁵Cl is enriched to exceed 76.01%, ⁴⁰Ca is enriched to exceed 96.95%, ⁴²Ca is enriched to exceed 0.65%, ⁴³Ca is enriched to exceed 0.65%, ⁵⁰V is enriched to exceed 0.25%, ⁵⁰Cr is enriched to exceed 4.25%, ⁵²Cr is enriched to exceed 85.01%, ⁵⁴Fe is enriched to exceed 5.85%, ⁵⁶Fe is enriched to exceed 91.8%, ⁵⁸Ni is enriched to exceed 68.10%, ⁶⁰Ni is enriched to exceed 26.51%, ⁶¹Ni is enriched to exceed 1.12%, ⁶³Cu is enriched to exceed 69.10%, ⁶⁴Zn is enriched to exceed 58.61%, ⁶⁶Zn is enriched to exceed 28.10%, ⁸⁴Sr is enriched to exceed 0.60%, ⁹²Mo is enriched to exceed 15.01%, ⁹⁴Mo is enriched to exceed 9.25%, ⁹⁵Mo is enriched to exceed 15.95%, ⁹⁶Mo is enriched to exceed 16.69%, ⁷⁴Se is enriched to exceed 0.90%, ⁷⁶Se is enriched to exceed 9.25%, ⁷⁷Se is enriched to exceed 7.65%, ⁷⁸Se is enriched to exceed 23.81%, ⁷⁰Ge is enriched to exceed 20.55%, ⁷²Ge is enriched to exceed 27.57%, ⁷³Ge is enriched to exceed 7.77%, ⁶⁹Ga is enriched to exceed 64.11%, ⁷⁹Br is enriched to exceed 50.70%, ⁸⁵Rb is enriched to exceed 72.20%, ¹⁰⁷Ag is enriched to exceed 51.61%, ¹³⁰Ba is enriched to exceed 0.12%, ¹³²Ba is enriched to exceed 0.12%, ¹³⁴Ba is enriched to exceed 2.42%, ¹³⁵Ba is enriched to exceed 6.60%, and/or ¹³⁶Ba is enriched to exceed 7.86%.

6. The method of claim 5, wherein the colloidal substance form includes at least one of sanitized water, distilled water, demineralized water, ionized water, deuterium-depleted water, pharmaceutically acceptable oil, natural polymer, synthetic polymer, carboxymethylcellulose, methylcellulose, hydroxypropyl cellulose, and/or a combination thereof.

7. The method of claim 5, wherein the pharmaceutically acceptable compound includes at least one non-steroidal anti-inflammatory drug selected from ibuprofen, aspirin, naproxen, diclofenac, etodolac, fenoprofen, flurbiprofen, indomethacin, meclofenamate, mefenamic acid, nabumetone, naproxen, oxaprozin, piroxicam, sulindac, tolmetin, a COX-2 inhibitor, or a combination thereof.

8. The method of claim 5, wherein the pharmaceutically acceptable compound includes at least one of an enzyme inhibitor, ATP-binding agent, solute-linked carrier, organic anion or cation, or non-organic anion or cation, a polymeric nanocarrier, a metal-organic framework, a lipid-based nanoparticle, a dendrimer, a nanostructured lipid carrier, a carbon nanotube, or a combination thereof.

9. The method of claim 5, wherein the salt includes at least one of an organic salt, an inorganic salt, or a chelating agent, or a combination thereof.

10. A method of reducing oxidative stress including administering a therapeutically effective amount to a subject in need thereof of at least one isotope-specific chemical element selected from ⁶Li, ¹²C, ¹⁰B, ²⁴Mg, ²⁵Mg, ²⁸Si, ²⁹Si, ³²S, ³³S, ³⁹K, ³⁵Cl, ⁴⁰Ca, ⁴²Ca, ⁴³Ca, ⁵⁰V, ⁵⁰Cr, ⁵²Cr, ⁵⁴Fe, ⁵⁶Fe, ⁵⁸Ni, ⁶⁰Ni, ⁶¹Ni, ⁶³Cu, ⁶⁴Zn, ⁶⁶Zn, ⁸⁴Sr, ⁹²Mo, ⁹⁴Mo, ⁹⁵Mo, ⁹⁶Mo, ⁷⁴Se, ⁷⁶Se, ⁷⁷Se, ⁷⁸Se, ⁷⁰Ge, ⁷²Ge, ⁷³Ge, ⁶⁹Ga, ⁷⁹Br, ⁸⁵Rb, ¹⁰⁷Ag, ¹³⁰Ba, ¹³²Ba, ¹³⁴Ba, ¹³⁵Ba, and/or ¹³⁶Ba, present in elemental form, atomic form, colloidal substance form, in form of pharmaceutically acceptable compound, or in form of a salt, wherein ⁶Li is enriched to exceed 10.06%, ¹²C is enriched to exceed 98.93%, ¹⁰B is enriched to exceed 21.01%, ²⁴Mg is enriched to exceed 80.01%, ²⁵Mg is enriched to exceed 10.01%, ²⁸Si is enriched to exceed 92.51%, ²⁹Si is enriched to exceed 5.01%, ³²S is enriched to exceed 95.03%, ³³S is enriched to exceed 0.91%, ³⁹K is enriched to exceed 93.51%, ³⁵Cl is enriched to exceed 76.01%, ⁴⁰Ca is enriched to exceed 96.95%, ⁴²Ca is enriched to exceed 0.65%, ⁴³Ca is enriched to exceed 0.65%, ⁵⁰V is enriched to exceed 0.25%, ⁵⁰Cr is enriched to exceed 4.25%, ⁵²Cr is enriched to exceed 85.01%, ⁵⁴Fe is enriched to exceed 5.85%, ⁵⁶Fe is enriched to exceed 91.8%, ⁵⁸Ni is enriched to exceed 68.10%, ⁶⁰Ni is enriched to exceed 26.51%, ⁶¹Ni is enriched to exceed 1.12%, ⁶³Cu is enriched to exceed 69.10%, ⁶⁴Zn is enriched to exceed 58.61%, ⁶⁶Zn is enriched to exceed 28.10%, ⁸⁴Sr is enriched to exceed 0.60%, ⁹²Mo is enriched to exceed 15.01%, ⁹⁴Mo is enriched to exceed 9.25%, ⁹⁵Mo is enriched to exceed 15.95%, ⁹⁶Mo is enriched to exceed 16.69%, ⁷⁴Se is enriched to exceed 0.90%, ⁷⁶Se is enriched to exceed 9.25%, ⁷⁷Se is enriched to exceed 7.65%, ⁷⁸Se is enriched to exceed 23.81%, ⁷⁰Ge is enriched to exceed 20.55%, ⁷²Ge is enriched to exceed 27.57%, ⁷³Ge is enriched to exceed 7.77%, ⁶⁹Ga is enriched to exceed 64.11%, ⁷⁹Br is enriched to exceed 50.70%, ⁸⁵Rb is enriched to exceed 72.20%, ¹⁰⁷Ag is enriched to exceed 51.61%, ¹³⁰Ba is enriched to exceed 0.12%, ¹³²Ba is enriched to exceed 0.12%, ¹³⁴Ba is enriched to exceed 2.42%, ¹³⁵Ba is enriched to exceed 6.60%, and/or ¹³⁶Ba is enriched to exceed 7.86%.

11. The method of claim 10, wherein the colloidal substance further includes at least one of ozonized water, distilled water, demineralized water, ionized water, and/or deuterium-depleted water, or a combination thereof.

12. The method of claim 10 further includes administering at least one of paracetamol, resveratrol, beta-carotene, vitamin B, vitamin C, vitamin D, vitamin E, glutathione, coenzyme Q10, caffein, and/or or a combination thereof.

13. The method of claim 10 further includes administering at least one of histidine, arginine, leucine, isoleucine, lysine, methionine, phenylalanine, tyrosine, threonine, tryptophan, valine, or a combination thereof.

14. The method of claim 10 further includes administering at least one of a proteinogenic amino acid or nonessential amino acid or a combination thereof.
